(19) United States
(12) Patent Application Publication
(10) Pub. No.: US 2012/0063953 A1
(43) Pub. Date: Mar. 15, 2012

(54) MEDICAL HEAT EXCHANGER, MANUFACTORING THEREOF AND ARTIFICIAL LUNG DEVICE

(75) Inventors: Hideki Izumida, Hiroshima (JP); Tomokazu Niitsuma, Hiroshima (JP)

(73) Assignee: JMS CO., LTD., Hiroshima-shi, Hiroshima (JP)

(21) Appl. No.: 13/321,037

(22) PCT Filed: Mar. 5, 2010
(86) PCT No.: PCT/JP2010/053645

§ 371 Date: Nov. 17, 2011

(30) Foreign Application Priority Data
Jun. 9, 2009 (JP) 2009-137981

(51) Int. Cl. A61M 1/36; F28F 9/02

(52) U.S. Cl. 422/44; 165/173

(57) ABSTRACT

A thin tube bundle (2) including a plurality of heat transfer thin tubes (1) is sealed by seal members (3a-3c) to form a blood channel (5) that crosses the heat transfer thin tubes. Heat transfer thin tube headers (6, 7) having an inlet port and an outlet port (6a, 7a) of heat medium liquid form flow chambers that contain ends of the thin tube bundle. The thin tube bundle is divided in a direction of the blood channel and forms a stack structure of thin tube bundle units (12a-12c). The flow chambers are partitioned into a plurality of flow compartments (13a, 13b, 14a, 14b) by partition walls (6b, 7b) to form a channel that allows heat medium liquid to pass through the respective thin tube bundle units successively via the flow compartments. An end of one of the thin tube bundle units on both sides of a border corresponding to the partition wall protrudes further than an end of the other thin tube bundle unit, and a side face of the partition wall contacts an side face of the protruding portion. Thus, the flow velocity of the heat medium liquid flowing through the heat transfer thin tubes is increased, and hence, heat exchange efficiency is enhanced while suppressing the increase in volume of the blood channel.

### TECHNICAL FIELD

The present invention relates to a heat exchanger, in particular, to a medical heat exchanger suitable for use in medical equipment such as an artificial lung device, a method for producing the heat exchanger, and an artificial lung device having the heat exchanger.

### BACKGROUND ART

In heart surgery, a cardiopulmonary bypass device is used when the heartbeat of a patient is caused to cease and it takes the place of the heart to perform the respiration and circulation functions during the cessation of the heartbeat. Further, during the surgery, in order to reduce the amount of oxygen to be consumed by the patient, it is necessary to lower the body temperature of the patient and maintain the lowered temperature. Therefore, the cardiopulmonary bypass device is provided with a heat exchanger for controlling the temperature of blood collected from the patient.

As such a medical heat exchanger, conventionally, a bellows tube type heat exchanger and a multitubular heat exchanger are known. Of them, the multitubular heat exchanger has an advantage of a higher heat exchange efficiency compared with that of the bellows tube type heat exchanger, because the multitubular heat exchanger can obtain a larger heat exchange area if the volume of the multitubular heat exchanger is the same as that of the bellows tube type heat exchanger.

A conventional exemplary multitubular heat exchanger described in Patent Document 1 will be described with reference to FIGS. 10A-10C. FIG. 10A is a top view of a multitubular heat exchanger, and FIG. 10B is a side view thereof. FIG. 10C is a perspective view illustrating a thin tube bundle module inside a housing of the heat exchanger, which is illustrated partially in a cross-section.

The heat exchanger includes a thin tube bundle 102 composed of a plurality of heat transfer thin tubes 101 allowing cool/warm water that is heat medium liquid to flow, seal members 103a-103c sealing the thin tube bundle 102, and a housing 104 containing these components.

A plurality of the heat transfer thin tubes 101 are arranged in parallel and stacked to form the thin tube bundle 102. As illustrated in FIGS. 10A and 10C, the seal member 103c at the center is provided with a blood channel 105 having a circular cross-section at the center in a longitudinal direction of the thin tube bundle 102. The blood channel 105 functions as a heat exchange channel for distributing blood that is liquid to be subjected to heat exchange so that the blood comes into contact with each outer surface of the heat transfer thin tubes 101. The seal members 103a, 103b at both ends respectively expose both ends of the thin tube bundle 102.

As illustrated in FIG. 10B, the housing 104 has a blood inlet port 106 for introducing blood into the housing 104 and a blood outlet port 107 for discharging the blood out of the housing 104, which are located at upper and lower ends of the blood channel 105. Further, gaps 108 are provided between the seal members 103a-103c respectively. The housing 104 is provided with leaked liquid discharge holes 109 corresponding to the gaps 108.

In the above-mentioned configuration, blood is allowed to flow in from the blood inlet port 106 and flow out of the blood outlet port 107 after passing through the blood channel 105. Simultaneously, as illustrated in FIGS. 10A and 10B, cool/warm water is allowed to flow in from one exposed end of the thin tube bundle 102 and flow out of the other exposed end thereof Thus, the heat exchange is performed between the blood and the cool/warm water in the blood channel 105.

The gaps 108 are provided for the purpose of detecting leakage when the blood or cool/warm water leaks due to seal leakage. More specifically, when leakage from the third seal member 103c occurs, the leaked blood appears in the gaps 108 and thus, the leakage can be detected. Further, even when the cool/warm water leaks due to the leakage from the first seal member 103a or the second seal member 103b, the leaked cool/warm water appears in the gaps 108, and thus, the leakage can be detected. The blood or cool/warm water having leaked in the gaps 108 is discharged out of the heat exchanger from the leaked liquid discharge holes 109.

### PRIOR ART DOCUMENT

### Patent Document

Patent Document 1: JP 2005-224301 A

### DISCLOSURE OF INVENTION

### Problem to be Solved by the Invention

There is a demand for the heat exchange efficiency of the above-mentioned multitubular heat exchanger to be enhanced further. This is because it is necessary to enhance the heat exchange efficiency in order to minimize the priming volume of blood in the blood channel 105 and further obtain sufficient heat exchange ability

In the case of a heat exchanger for an artificial lung considered by the inventors of the present invention, it was found that the heat exchange efficiency practically is desired to be 0.43 or more. The heat exchange area required for achieving the target value was 0.014 m² at a blood flow rate of 2 L/min. If this is applied to a configuration in which the ability of the heat exchanger is enhanced to a blood flow rate of 7 L/min, as a result of heat exchange area simulation, it was found that a heat exchange area of 0.049 m² is required for obtaining a heat exchange efficiency of 0.43 or more. Herein, the heat exchange efficiency is defined by the following expression.

Heat exchange efficiency=(T_{BOUT}−T_{BIN})/(T_{WIN}−T_{BIN})

T_{BIN}: blood inflow side temperature

T_{BOUT}: blood outflow side temperature

T_{WIN}: heat medium (water) inflow side temperature

For example, the following is found: when using the heat transfer thin tubes 101 with an outer diameter of 1.25 mm, if the stacking number (number of thin tube layers) of the heat transfer thin tubes 101 is set at six, a heat exchange area of 0.057 m² can be obtained. However, when the heat exchange efficiency was measured with an opening diameter of the blood channel 105 set at 70 mm, using a heat exchange module including the thin tube bundle 102 with such a six-layered configuration, only a value much lower than the target value (i.e., 0.24) was obtained.

Then, a heat exchange module was produced in which the heat transfer thin tubes 101 with an outer diameter of 1.25 mm were used, an opening diameter of the blood channel 105 was set at 70 mm, and the number of thin tube layers was increased variously, and the heat exchange efficiency was measured using the module. As a result, it was found that, in order to achieve a heat exchange efficiency of 0.43, it is necessary to set the number of thin tube layers at 18 or more. However, if the number of thin tube layers is set at 18 under the above-mentioned conditions, the blood priming volume in the blood channel becomes 42.3 mL. This exceeds 30 mL, which is a desired value of the blood priming volume. In order to set the blood priming volume at 30 mL or less, the number of thin tube layers should be set at 13 or less according to calculations.

Thus, it is difficult to obtain the desired heat exchange efficiency merely by increasing a heat exchange area. Therefore, the cause that seems to decrease heat exchange efficiency was analyzed. Consequently, as the cause for decreasing heat exchange efficiency, it was found that a flow velocity of cool/warm water flowing through lumens of the heat transfer thin tubes 101 has large influence. This is considered to be caused by the influence of a flow velocity of cool/warm water on a change in a film resistance.

An object of the present invention is to provide a medical heat exchanger capable of enhancing heat exchange efficiency while reducing the volume of a heat exchange region by controlling the flow of heat medium liquid in lumens of heat transfer thin tubes appropriately.

### Means for Solving Problem

A medical heat exchanger of the present invention includes: a thin tube bundle in which a plurality of heat transfer thin tubes for allowing heat medium liquid to flow through a lumen are arranged and stacked; a seal member sealing the thin tube bundle while allowing both ends of the heat transfer thin tubes to be exposed and forming a blood channel that crosses the heat transfer thin tubes for allowing blood to flow therethrough so that the blood comes into contact with an outer surface of each of the heat transfer thin tubes; a housing containing the seal member and the thin tube bundle and provided with an inlet port and an outlet port for the blood positioned respectively at both ends of the blood channel; and a pair of heat transfer thin tube headers forming flow chambers that respectively contain both ends of the thin tube bundle and having an inlet port and an outlet port for the heat medium liquid.

In order to solve the above-described problem, the thin tube bundle is divided into a plurality of stages in a flow direction of the blood channel, and functions as a stack structure of thin tube bundle units of the respective stages, each stage being composed of members of the plurality of the heat transfer thin tubes. At least one of the flow chambers is partitioned, by a partition wall provided so as to correspond to a border between the thin tube bundle units, into a plurality of flow compartments so that each flow compartment contains an end of one or two stages of the thin tube bundle units, whereby a channel is formed such that the heat medium liquid flowing in from the inlet port is introduced via any one of the flow compartments so as to pass through the plurality of stages of the thin tube bundle units successively and flows out of the outlet port via another of the flow compartments. An end of one of the thin tube bundle units that is positioned on both sides of the border corresponding to the partition wall protrudes further than an end of the other thin tube bundle unit, and a side face of the partition wall contacts an side face of the protruding thin tube bundle unit, whereby the flow compartments on both sides of the partition wall are separated from each other.

### Effect of the Invention

According to the above-mentioned configuration of the medical heat exchanger of the present invention, heat medium liquid successively passes through a plurality of groups of thin tube bundle units into which the thin tube bundle is divided, and hence, the flow velocity of cool/warm water flowing through the heat transfer thin tubes of each thin tube bundle unit can be increased. Consequently, the heat exchange efficiency can be enhanced while the film resistance at the inner walls of the heat transfer thin tubes is reduced to suppress the increase in volume of a heat exchange region.

Further, a plurality of flow compartments therefor can be formed by a simple configuration in which an end of one of the thin tube bundle units of stages on both sides of the border corresponding to the partition wall protrudes, and a side face of the partition wall contacts the protruding side face. Thus, an interval between the thin tube bundle units can be minimized, thereby suppressing a blood priming volume in the heat exchange region to the minimum.

### BRIEF DESCRIPTION OF DRAWINGS

FIG. 1A is a top view illustrating a configuration of a medical heat exchanger in Embodiment 1

FIG. 1B is a cross-sectional view taken along the line A-A in FIG. 1A of the medical heat exchanger.

FIG. 1C is a cross-sectional view taken along the line B-B in FIG. 1A of the medical heat exchanger.

FIG. 2A is an enlarged cross-sectional view illustrating an important portion of the medical heat exchanger.

FIG. 2B is an enlarged cross-sectional view illustrating another important portion of the medical heat exchanger.

FIG. 3A is a perspective view illustrating a thin tube bundle module in which thin tube bundle units are stacked, which is used in the medical heat exchanger.

FIG. 3B is a front view of the module.

FIG. 4A is a perspective view of a unit thin tube row constituting the thin tube bundle unit contained in the module.

FIG. 4B is a front view of the unit thin tube row.

FIG. 5 is a diagram illustrating a relationship between a form of division of a thin tube bundle and a heat exchange coefficient.

FIG. 6 is a diagram illustrating a relationship between a turnback structure of the thin tube bundle and the heat exchange coefficient.

FIG. 7A is an enlarged cross-sectional view illustrating an important portion in another form of the medical heat exchanger in Embodiment 1.

FIG. 7B is an enlarged cross-sectional view illustrating another important portion of the medical heat exchanger.

FIG. 8 is an enlarged cross-sectional view illustrating an important portion in still another form of the medical heat exchanger in Embodiment 1.

FIG. 9 is a cross-sectional view illustrating an artificial lung device in Embodiment 2.

FIG. 10A is a top view illustrating a configuration of a heat exchanger in a conventional example.

FIG. 10B is a side view illustrating the configuration of the same heat exchanger.

FIG. 10C is a perspective view illustrating a partial cross-section of a thin tube bundle module in the same heat exchanger.

### DESCRIPTION OF PREFERRED EMBODIMENT

### Description of the Invention

A medical heat exchanger of the present invention can take the following forms based on the above-mentioned configuration.

It is preferable that, of the thin tube bundle units of the stages on the both sides of the border corresponding to the partition wall, an end of the thin tube bundle unit placed on a side where the heat medium liquid is introduced in the channel of the heat medium liquid protrudes further than an end of the thin tube bundle unit placed on a side where the heat medium liquid is discharged. In this case, the partition wall comes into contact with a side face of the thin tube bundle unit placed on the side where the heat medium liquid is introduced. Thus, the heat medium liquid flowing into the heat transfer thin tube 1 does not flow in a direction colliding with respect to a contact face between the protruding portion of the thin tube bundle unit and the partition wall.

Further, it is preferable that a side face portion of the partition wall contacting a side face of the thin tube bundle unit forms a taper, which is made thinner toward an inside of the heat transfer thin tubes. Thus, a pressing force acts between the side face of the thin tube bundle unit and the tapered face of the partition wall, thereby improving sealing integrity between the both side faces.

Further, it is preferable that the heat transfer thin tube headers are formed so that the heat medium liquid successively passes from the thin tube bundle unit in a lower stage placed on a downstream side of the blood channel to the thin tube bundle unit in an upstream stage placed on an upstream side. This causes the flow of the heat medium liquid to be a counterflow with respect to the flow of liquid to be subjected to heat exchange, which is advantageous for enhancing the heat exchange efficiency

Further, it is preferable that the blood channel is formed in a cylindrical shape whose circumference is sealed with the seal member.

It is possible to configure an artificial lung device that includes the heat exchanger having one of the above-described configurations; and an artificial lung having a blood channel that crosses a gas channel so as to perform gas exchange. The heat exchanger and the artificial lung are stacked, and the blood channel of the heat exchanger and the blood channel of the artificial lung communicate with each other.

Hereinafter, a medical heat exchanger in an embodiment of the present invention will be described with reference to the drawings. The following embodiments are exemplary applications to an artificial lung device and will be described exemplifying a heat exchanger used for adjusting the temperature of blood collected from a patient

### Embodiment 1

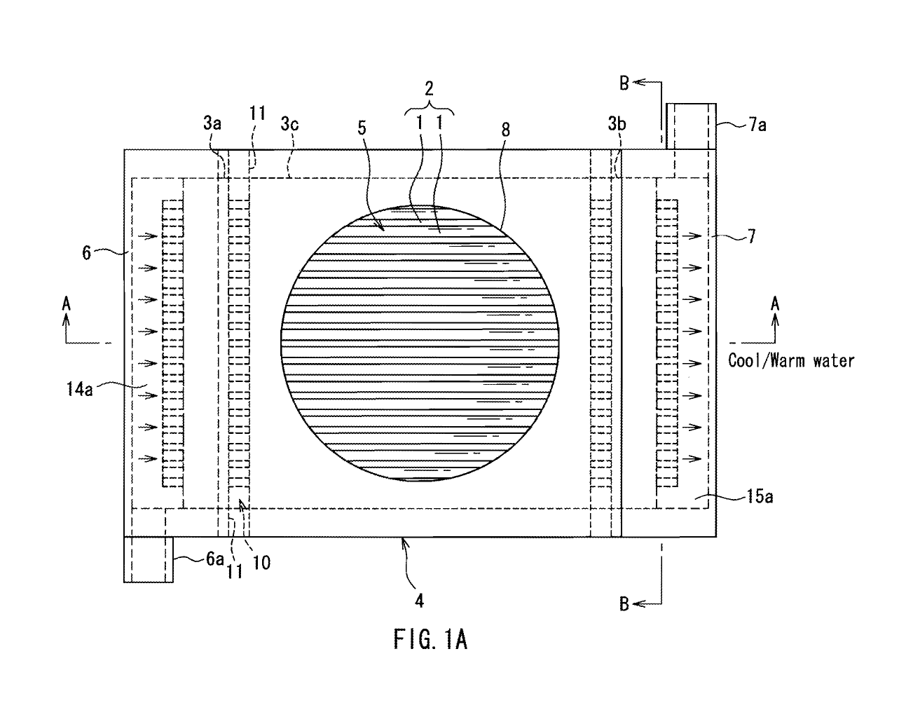

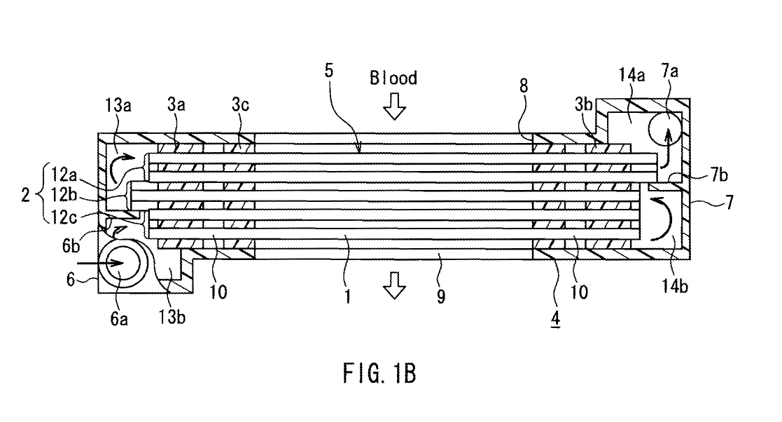

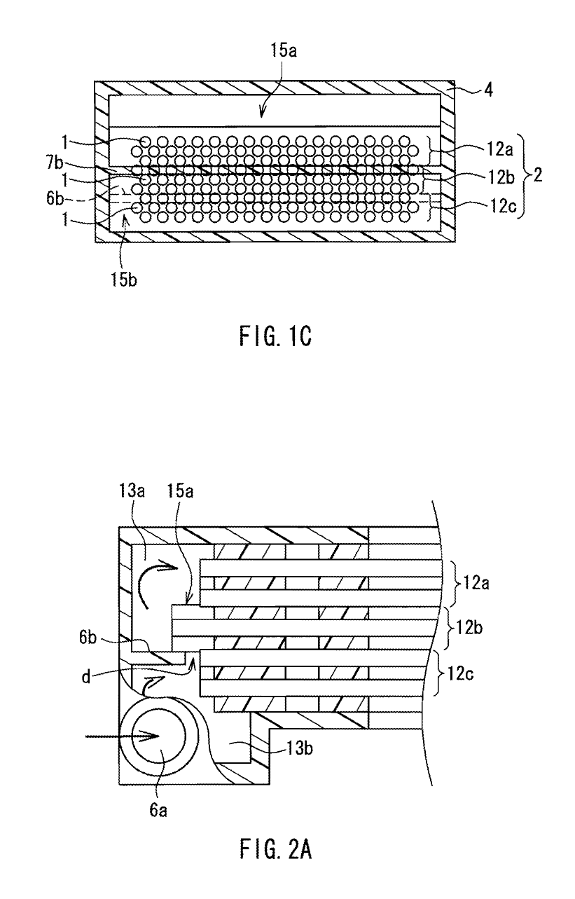

FIG. 1A is a plan view illustrating a medical heat exchanger in Embodiment 1. FIG. 1B is a cross-sectional view taken along the line A-A in FIG. 1A, and FIG. 1C is a cross-sectional view taken along the line B-B in FIG. 1A. The heat exchanger includes a thin tube bundle 2 composed of a plurality of heat transfer thin tubes 1 for distributing cool/warm water as heat medium liquid, seal members 3a-3c sealing the thin tube bundle 2, and a housing 4 containing these components.

A plurality of the heat transfer thin tubes 1 are arranged in parallel and stacked to form the thin tube bundle 2, and cool/warm water is allowed to flow through a lumen of each heat transfer thin tube 1. A blood channel 5 having a circular cross-section is formed in a center portion in a longitudinal direction of the thin tube bundle 2 in the seal member 3c at the center, and functions as a heat exchange region for letting blood flow as the liquid to be subjected to heat exchange. When the blood passing through the blood channel 5 comes into contact with each outer surface of the heat transfer thin tube 1, heat exchange is performed. The seal members 3a, 3b at both ends expose both ends of the thin tube bundle 2.

The housing 4 has heat transfer thin tube headers, i.e., a cool/warm water inlet header 6 for introducing cool/warm water and a cool/warm water outlet header 7 for discharging the cool/warm water, facing both ends of the thin tube bundle 2. Further, as illustrated in FIG. 1B, the housing 4 is provided with a blood inlet port 8 and a blood outlet port 9, positioned at upper and lower ends of the blood channel 5. The cool/warm water inlet header 6 and the cool/warm water outlet header 7 respectively are provided with a cool/warm water inlet port 6a and a cool/warm water outlet port 7a. Further, gaps 10 are provided respectively between the seal members 3a-3c as in the conventional example, and the housing 4 is provided with leaked liquid discharge holes 11 corresponding to the gaps 10.

As illustrated in FIG. 1B, the cool/warm water inlet header 6 and the cool/warm water outlet header 7 form flow chambers that are spaces respectively containing both ends of the thin tube bundle 2 exposed from the seal members 3a, 3b at both ends. The flow chamber on the left side is partitioned into an upper flow compartment 13a and a lower flow compartment 13b, and the flow chamber on the right side is partitioned into an upper flow compartment 14a and a lower flow compartment 14b. Thus, the cool/warm water that is to be introduced and discharged all flows via the flow compartments formed by the cool/warm water inlet header 6 and the cool/warm water outlet header 7.

According to the present embodiment, as illustrated in FIG. 1B, the thin tube bundle 2 is divided into three stages in a flow direction of the blood channel 5 and functions as a stack structure of the first to third thin tube bundle units 12a-12c, each stage including the three-layered heat transfer thin tubes 1. Both ends of the first to third thin tube bundle units 12a-12c respectively correspond to the upper flow compartments 13a, 14a and the lower flow compartments 13b, 14b.

The upper flow compartment 13a and the lower flow compartment 13b on the left side are separated by a partition wall 6b. Left ends of the first and second thin tube bundle units 12a, 12b are placed in the upper flow compartment 13a, and a left end of the third thin tube bundle unit 12c is placed in the lower flow compartment 13b. More specifically, the partition wall 6b is placed at a border portion between the second thin tube bundle unit 12b and the third thin tube bundle unit 12c. Similarly, the upper flow compartment 14a and the lower flow compartment 14b on the right side are separated by a partition wall 7b. A right end of the first thin tube bundle unit 12a is placed in the upper flow compartment 14a, and right ends of the second and third thin tube bundle units 12b, 12c are placed in the lower flow compartment 14b. More specifically, the partition wall 7b is placed at a border portion between the first thin tube bundle unit 12a and the second thin tube bundle unit 12b.

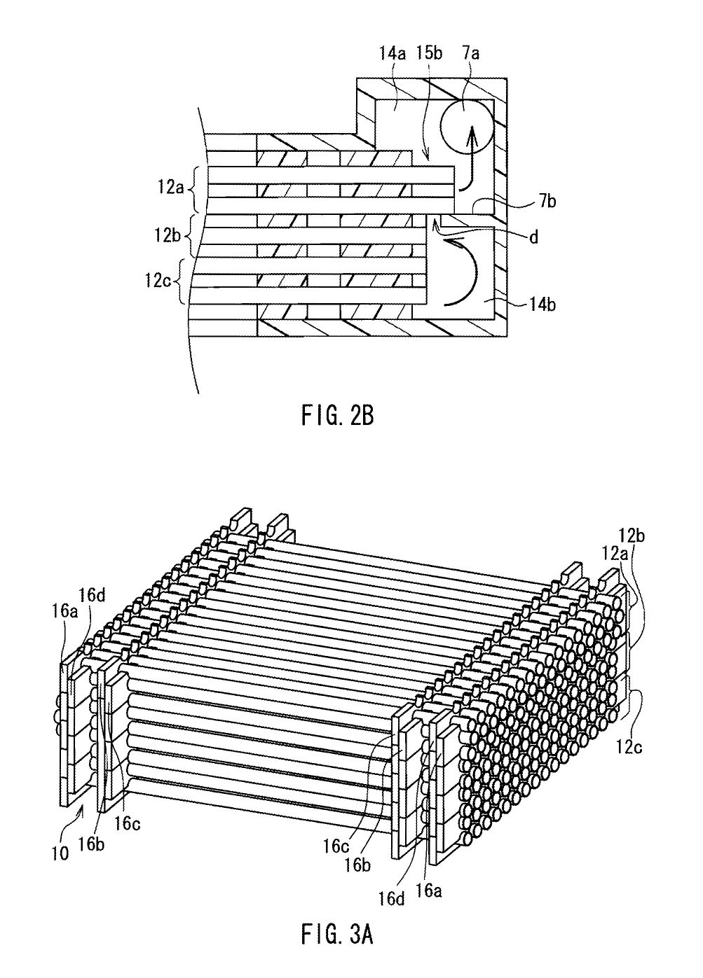

In order to separate the upper flow compartment 13a and the lower flow compartment 13b on the left side in the drawings by the partition wall 6b, the left end of the second thin tube bundle unit 12b forms a protruding portion 15a that protrudes further than the left end of the third thin tube bundle unit 12c as illustrated in an enlarged state in FIG. 2A. A side face of the partition wall 6b contacts a side face of the protruding portion 15a of the second thin tube bundle unit 12b. Thus, a practically effective liquid-tight structure is formed at a border between the side face of the protruding portion 15a and the side face of the partition wall 6b. An interval d is provided between a left end face of the third thin tube bundle unit 12c and an end of the partition wall 6b.

Herein, the practically effective liquid-tight structure means that, when cool/warm water introduced from the cool/warm water inlet port 6a to the lower flow compartment 13b flows into the third thin tube bundle unit 12c, the flow that is leaked into the upper flow compartment 13a from the border portion between the both side faces in the protruding portion 15a is controlled to the extent as not to cause a practical problem. Since the leakage of the cool/warm water into the upper flow compartment 13a does not cause problems such as influences on blood, a hermetical structure that perfectly blocks liquid is not required. Therefore, the side face of the partition wall 6b need not contact the side face of the protruding portion 15a, and a clearance may be present to some extent. However, since such a leakage may decrease the heat exchange efficiency, it is desirable that the clearance is suppressed within a set range.

Similarly, in order to separate the upper flow compartment 14a and the lower flow compartment 14b on the right side by the partition wall 7b, the right end of the first thin tube bundle unit 12a forms a protruding portion 15b that protrudes further than the right end of the second thin tube bundle unit 12b as illustrated in an enlarged state in FIG. 2B. A side face of the partition wall 7b contacts a side face of the protruding portion 15b of the first thin tube bundle unit 12a. Thus, a practically effective liquid-tight structure is formed at a border portion between the side face of the protruding portion 15b and the side face of the partition wall 7b. An interval d is provided between a right end face of the second thin tube bundle unit 12b and an end of the partition wall 7b.

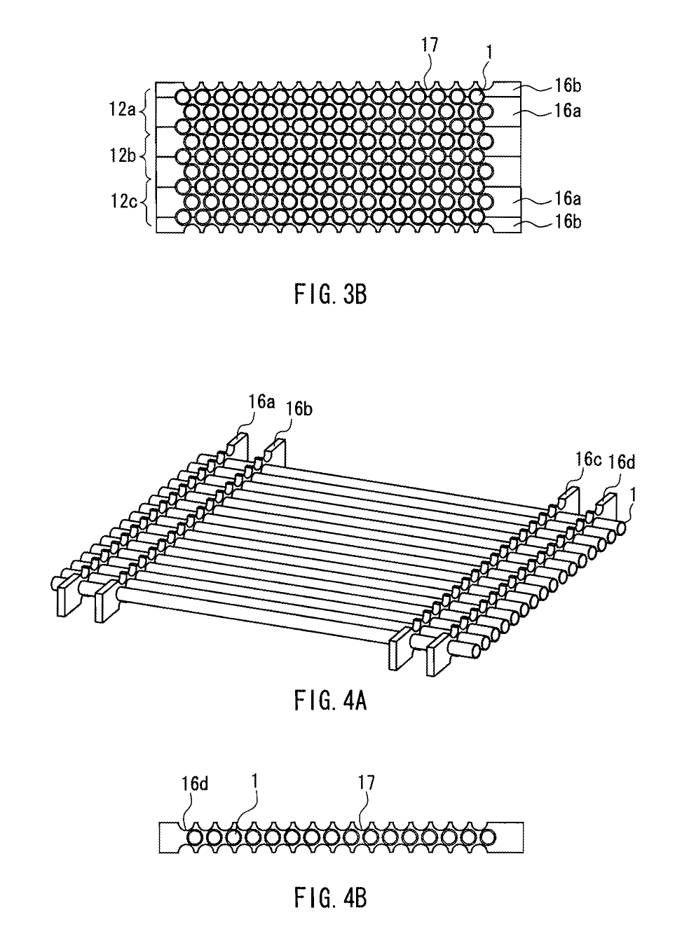

Next, an example of detailed structures of the first to third thin tube bundle units 12a-12c will be described with reference to FIGS. 3A, 3B, 4A and 4B. FIG. 3A is a perspective view illustrating a form of a thin tube bundle module in which the heat transfer thin tubes 1 are stacked to form the thin tube bundle 2. For convenience of illustration, the size in a vertical direction is illustrated in an enlarged state, compared with FIG. 1B. In the subsequent other figures, the size in the vertical direction will be illustrated in an enlarged state similarly. FIG. 3B is a front view of the module.

As illustrated in FIGS. 3A and 3B, the thin tube bundle units 12a-12c respectively have a configuration in which a plurality of heat transfer thin tubes 1 are bound by thin tube row holding members 16a-16d arranged at four portions in an axis direction of the heat transfer thin tubes 1. One set of the thin tube row holding members 16a-16d binds one row (one layer) of a thin tube row. The bound state is illustrated in the perspective view of FIG. 4A. FIG. 4B is a front view thereof.

A plurality of the heat transfer thin tubes 1 (16 in the example of FIG. 4A) arranged in a row in parallel to each other are held by the thin tube row holding members 16a-16d, and thus, one layer of a heat transfer thin tube row is formed. The thin tube row holding members 16a-16d respectively are formed in a band shape traversing the heat transfer thin tubes 1, and the heat transfer thin tubes 1 pass through the thin tube row holding members 16a-16d.

The heat transfer thin tube row in such a form can be formed by so-called insert molding of injecting resin into a die in which a plurality of the heat transfer thin tubes 1 are arranged to form the thin tube row holding members 16a-16d. Upper and lower surfaces of the thin tube row holding members 16a-16d are provided with a plurality of thin tube receiving concave portions 17 in which the heat transfer thin tubes 1 in another adjacent heat transfer thin tube row can be fitted.

The thin tube bundle units 12a-12c illustrated in FIG. 3A respectively are formed by stacking three layers of the row of the heat transfer thin tubes 1 in FIG. 4A. Note here that an interval between the first thin tube bundle unit 12a and the second thin tube bundle unit 12b is the same as intervals between the heat transfer thin tubes 1 in the thin tube bundle units 12a and 12b. The same applies to an interval between the second thin tube bundle unit 12b and the third thin tube bundle unit 12c. In other words, the configuration of the module composed of the thin tube bundle units 12a-12c is the same as the structure formed by simply stacking nine layers of the row of the heat transfer thin tubes 1 in FIG. 4A.

For stacking the row of the heat transfer thin tubes 1 in FIG. 4A, the heat transfer thin tubes 1 constituting each heat transfer thin tube row are fitted in the thin tube receiving concave portions 17 provided in the thin tube row holding members 16a-16d in upper and lower adjacent other heat transfer thin tube rows. Therefore, the thin tube row holding members 16a-16d are placed so as to be shifted from each other alternately for the respective upper and lower adjacent layers. Further, the thin tube row holding members 16a-16d are placed as a pair in each end region of the heat transfer thin tubes 1. More specifically, the thin tube row holding members 16a, 16b are placed close to each other at one end and the thin tube row holding members 16c, 16d are placed close to each other at the other end. Due to such an arrangement, the gaps 10 illustrated in FIG. 1B, etc. are formed between the thin tube row holding members 16b, 16d at both ends.

In use of the heat exchanger having the above-described configuration, as illustrated in FIGS. 1A and 1B, the blood is allowed to flow in the blood channel 5 from the blood inlet port 8 and flow out of the blood outlet port 9. Simultaneously, the cool/warm water is allowed to flow in the thin tube bundle 2 from the cool/warm water inlet header 6 and flow out of the cool/warm water outlet header 7. Thus, heat exchange is performed between the blood and the cool/warm water in the blood channel 5.

By this heat exchanger, the following functions and effects can be obtained. That is, cool/warm water introduced from the cool/warm water inlet port 6a on the left side to the lower flow compartment 13b of the cool/warm water inlet header 6 flows through lumens of the heat transfer thin tubes 1 of the third thin tube bundle unit 12c rightward and flows in the lower flow compartment 14b of the cool/warm water outlet header 7 on the right side. Further, the cool/warm water enters the heat transfer thin tubes 1 of the second thin tube bundle unit 12b and flows therethrough leftward to reach the upper flow compartment 13a of the cool/warm water inlet header 6. Then, the cool/warm water enters the heat transfer thin tubes 1 of the first thin tube bundle unit 12a and flows therethrough rightward to reach the upper flow compartment 14a of the cool/warm water outlet header 7 and flow out of the cool/warm water outlet port 7a.

Thus, the cool/warm water inlet header 6 and the cool/warm water outlet header 7 are configured so that the cool/warm water to be introduced passes through three stages of the third to first thin tube bundle units 12c-12a successively. The configuration in which the cool/warm water to be introduced passes through a plurality of groups of divided thin tube bundle units successively will be referred to as a “divided flow” hereinafter. In contrast, the configuration in which the cool/warm water to be introduced flows into all the heat transfer thin tubes 1 at a time in the cool/warm water inlet header 6 as in the conventional example will be referred to as a “simultaneous flow”.

The channel cross-sectional area through which cool/warm water passes becomes smaller as a result of adopting the divided flow. Therefore, assuming that the volume flow rate of cool/warm water is the same, the flow velocity of the cool/warm water flowing through each heat transfer thin tube 1 of the first to third thin tube bundle units 12a-12c can be increased, compared with that of the simultaneous flow. This can reduce the film resistance in an inner wall of the heat transfer thin tube 1 to enhance heat exchange efficiency. In the conventional simultaneous flow, although the heat exchange efficiency can be enhanced by increasing the supply volume flow rate (or flow velocity) from the supply source of cool/warm water, it actually is difficult to increase the flow velocity of the supply source of cool/warm water on a medical facility side. Therefore, enhancing the heat exchange efficiency as in the present embodiment is very effective from the practical point of view.

Further, the cross-sectional configuration illustrated in FIG. 1B adopts a turnback structure in a vertical direction (perpendicular direction), i.e., a structure in which the thin tube bundle 2 is divided in a flow direction of blood (i.e., a vertical direction) to form a plurality of stages of thin tube bundle units. Further, the cool/warm water flows from the thin tube bundle unit 12c in the lowest stage placed on the downstream side of the blood channel 5 to the upstream stage through the thin tube bundle unit 12b and the thin tube bundle unit 12a successively. This means that the flow of the cool/warm water is formed to be a counterflow with respect to a blood flow, which is effective for obtaining higher heat exchange efficiency.

In order to form the turnback structure in the vertical direction as in the present embodiment, it is necessary that the flow chamber of the cool/warm water inlet header 6 be partitioned into the upper flow compartment 13a and the lower flow compartment 13b by the partition wall 6b, and the flow chamber of the cool/warm water outlet header 7 be partitioned into the upper flow compartment 14a and the lower flow compartment 14b by the partition wall 7b.

For this, a structure in which the protruding portions 15a and 15b respectively are provided at the left end of the second thin tube bundle unit 12b and the right end of the first thin tube bundle unit 12a as illustrated in FIGS. 2A and 2B is effective. Thus, the partition walls 6b and 7b be placed without providing any unnecessary intervals between the respective stages of the first to third thin tube bundle units 12a-12c. In other words, the intervals between the respective stages of the first to third thin tube bundle units 12a-12c can be the same as the stack interval of the heat transfer thin tubes 1 in the thin tube bundle units. Therefore, the thickness of the stack structure of the first to third thin tube bundle units 12a-12c can be minimized, thereby suppressing the blood priming volume in the blood channel 5 to the minimum.

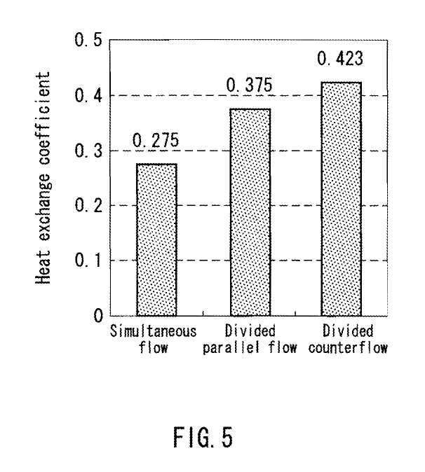

FIG. 5 illustrates the results obtained by conducting an experiment regarding the effect that the heat exchange efficiency is enhanced by the divided flow. The “divided parallel flow” and the “divided counterflow” in FIG. 5 indicate the case of the divided flow according to the present embodiment. The “divided counterflow” is the case where the thin tube bundle is divided along a direction of the blood flow and the flow of the heat medium liquid is set to be a counterflow as illustrated in FIG. 1B. The “divided parallel flow” refers to the case where the flow of the heat medium liquid is set to form a parallel flow whose direction is the same as that of the blood flow, although the form of division is the same In both the cases, an opening diameter of the blood channel 5 was set at 70 mm, and the number of layers of the heat transfer thin tubes 1 was set at 12.

It is understood from FIG. 5 that the heat exchange efficiency in the case of the divided parallel flow and the divided counterflow, both of which are a divided flow, is higher than that of the simultaneous flow. The reasons for this are as follows. Since the flow velocity of the cool/warm water flowing through the heat transfer thin tubes 1 is larger in the divided flow, the film resistance is reduced. Further in the case of the divided counterflow, the difference in temperature between the heat medium liquid and the blood can be kept high even on the blood downstream side, and as a result, the heat exchange efficiency is higher than that in the case of the divided parallel flow. The heat exchange efficiency in the case of the divided parallel flow is larger by 36%, and the heat exchange efficiency in the case of the divided counterflow is larger by 54%, compared with that in the case of the simultaneous flow.

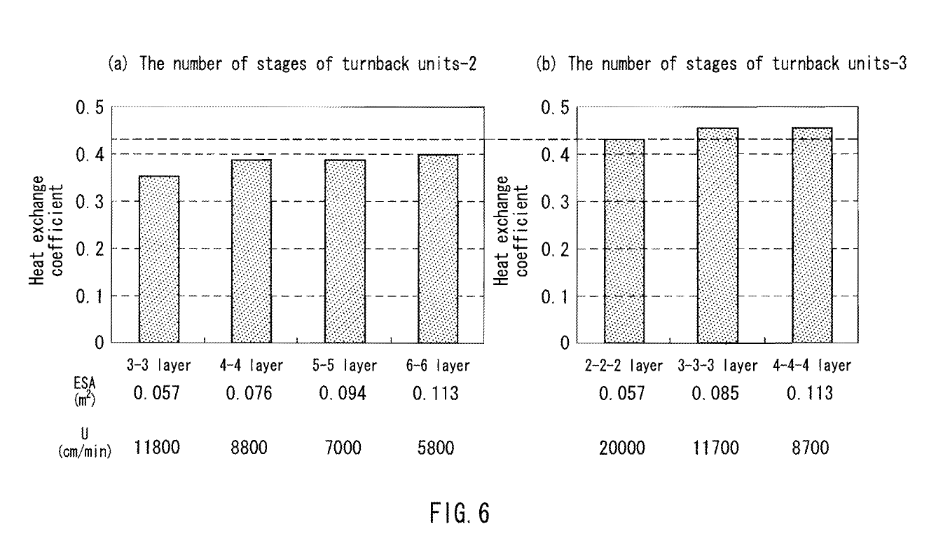

Next, FIG. 6 illustrates the results obtained by considering the appropriate number of layers of the thin tube bundle units and the appropriate number of layers of the heat transfer thin tubes 1 constituting each thin tube bundle unit in the case where the thin tube bundle 2 is divided in a vertical direction to form a plurality of layers of thin tube bundle units.

In FIG. 6, (a) illustrates the measurement results of heat exchange efficiency in the case where the number of stages of the thin tube bundle units is two, i.e., the number of stages at which the flow of the cool/warm water is turned back is two, and the heat transfer thin tubes constituting the thin tube bundle unit in each stage is three layers (number of stacked layers), four layers, five layers, and six layers. In FIG. 6, (b) illustrates the measurement results of the heat exchange efficiency in the case where the number of stages of the turnback thin tube bundle units is three, and the heat transfer thin tubes constituting the thin tube bundle unit in each stage is two layers, three layers, and four layers. ESA and U illustrated in a lower portion of a horizontal axis indicate an effective surface area and a flow velocity of a heat medium, respectively. It is understood from FIG. 6 that a higher heat exchange efficiency is likely to be obtained in the case (b) where the number of stages of the turnback thin tube bundle units is three, compared with the case (a) where the number of stages is two.

When the number of stages of the turnback thin tube bundle units is three, the heat exchange efficiency is slightly degraded in the case where the number of layers of the heat transfer thin tubes constituting a thin tube bundle unit is two, ie., a 2-2-2 layer structure at a left end in (b) of FIG. 6, compared with the case where the number of layers is three and four. However, high heat exchange efficiency can be obtained, relative to the case of two stages. Further, the total number of layers of the heat transfer thin tubes in three stages is six, and compared with a 3-3 layer structure in two stages having the same number of heat transfer thin tube layers, a sufficiently high heat exchange efficiency is obtained. The same number of layers of the heat transfer thin tubes means that a blood priming volume is substantially the same. Thus, it is understood that the heat exchange efficiency can be enhanced while the blood priming volume is suppressed according to the 2-2-2 layer structure.

It also is understood that no significant difference is found in heat exchange efficiency between the three and four layers of the heat transfer thin tubes constituting a thin tube bundle unit, when the number of stages is three. Four or more stages are excessive for performance, and in this case, a volume flow rate does not increase due to an increase in a pressure loss. Considering this result, it is understood that the most preferred structure from the practical point of view can be obtained when the thin tube bundle units, each being formed of three layers of heat transfer thin tubes, are stacked in three stages.

Further, in the case of an odd-number turnback structure as in a three-stage turnback structure, the cool/warm water inlet port 6a and the cool/warm water outlet port 7a can be provided at both ends of the thin tube bundle 2, and hence, the port layout has a good balance.

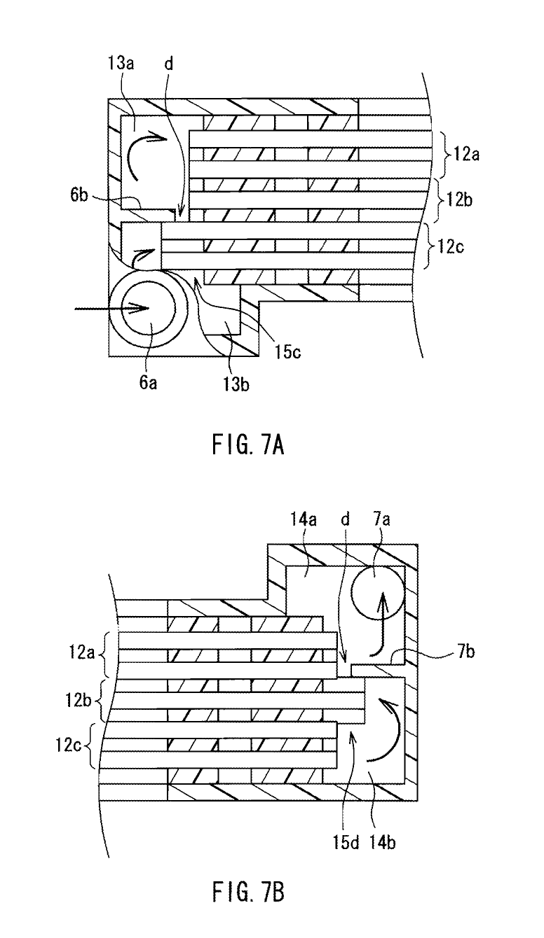

The structure for separating the upper flow compartment 13a and the lower flow compartment 13b by the partition wall 6b illustrated in FIG. 2A can be changed to a structure illustrated in FIG. 7A. Further, the structure for separating the upper flow compartment 14a and the lower flow compartment 14b by the partition wall 7b illustrated in FIG. 2B can be changed to a structure illustrated in FIG. 7B.

In the structure illustrated in FIG. 2A, the left end of the second thin tube bundle unit 12b forms the protruding portion 15a that protrudes further than the left end of the third thin tube bundle unit 12c. On the other hand, in the structure illustrated in FIG. 7A, the left end of the third thin tube bundle unit 12c forms a protruding portion 15c that protrudes further than the left end of the second thin tube bundle unit 12b. A side face of the partition wall 6b contacts an upper side face of the protruding portion 15c, and a practically effective liquid-tight structure is formed at a border between the both side faces. An interval d is provided between a left end face of the second thin tube bundle unit 12b and the end of the partition wall 6b.

Further, in the structure illustrated in FIG. 2B, the right end of the first thin tube bundle unit 12a forms the protruding portion 15b that protrudes further than the right end of the second thin tube bundle unit 12b. On the other hand, in the structure illustrated in FIG. 7B, the right end of the second thin tube bundle unit 12b forms a protruding portion 15d that protrudes further than the right end of the first thin tube bundle unit 12a. A side face of the partition wall 7b contacts an upper side face of the protruding portion 15d, and a practically effective liquid-tight structure is formed at a border between the both side faces. An interval is provided between a right end face of the first thin tube bundle unit 12a and the end of the partition wall 7b.

Note here that liquid leakage between the flow compartments is less likely to our in the structure illustrated in FIGS. 2A and 2B as compared with the structure illustrated in FIGS. 7A and 7B. This is because, in the structure of FIGS. 7A and FIG. 7B, the flow of heat medium liquid flowing out of the heat transfer thin tube 1 collides with the contact faces between the protruding portions of the thin tube bundle units and the partition walls 6b, 7b, whereas such a flow does not our in the structure of FIGS. 2A and 2B.

For these reasons, the structure illustrated in FIGS. 2A and 2B has a higher allowance for the presence of a clearance between the side face of the protruding portion 15a and the side face of the partition wall 6b. In other words, in order to suppress the leakage of cool/warm water into the upper flow compartment 13a within a range that does not cause a problem and to maintain the heat exchange efficiency within a set range, a larger clearance is allowed in the structure of FIGS. 2A and 2B as compared with the structure of FIGS. 7A and 7B. Therefore, the design and production of the structure of FIGS. 2A and 2B are easy.

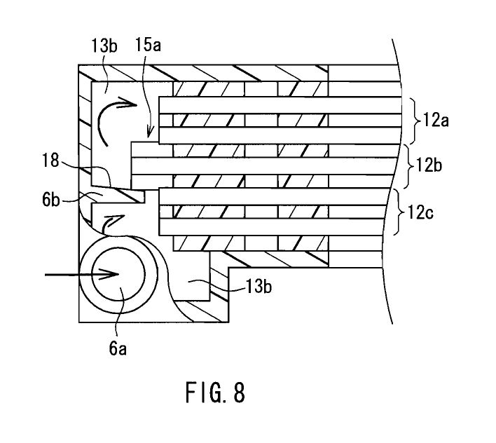

Further, in the configurations illustrated in FIGS. 2A, 2B and 7A, 7B, it is desirable that the side face portions of the partition walls 6b, 7b have a tapered shape as illustrated in FIG. 8. In other words, the side face portion of the partition wall 6b contacting the side face of the second thin tube bundle unit 12b forms a tapered face 18, which is made to be thinner toward the inside of the heat transfer thin tubes 1. When a positional relationship between the side face of the second thin tube bundle unit 12b and the tapered face 18 is set appropriately, a pressing force acts between the side face of the second thin tube bundle unit 12b and the tapered face 18 when they are assembled, thereby improving sealing integrity between the both side faces.

Although not illustrated in the above-mentioned figures, the housing 4 can be configured, for example, in such a manner that the housing 4 is separated into a housing bottom portion and a housing upper portion, which are combined with each other with the thin tube bundle 2 and the like contained therein. Alternatively, the housing 4 can be configured in such a manner that the housing 4 contains only the thin tube bundle 2 and the seal members 3a-3c, while the cool/warm water inlet header 6 and the cool/warm water outlet header 7 are separated from the housing 4.

The above description refers to the structures of the cool/warm water inlet header and the cool/warm water outlet header in the case where the thin tube bundle units have three stages. However, the cool/warm water inlet header and the cool/warm water outlet header can be configured similarly with ease even with another number of stages. More specifically, as a first setting, flow compartments are provided in the cool/warm water inlet header and the cool/warm water outlet header so as to correspond to one of the stages of the thin tube bundle units positioned at an upstream side end or a downstream side end. Further, the flow compartments are provided so as to correspond respectively to the thin tube bundle units of the every other pairs of the stages. Each of the inlet port and the outlet port is provided with respect to the flow compartment corresponding to the first stage of the thin tube bundle unit. This forms a channel in such a manner that heat medium liquid flowing in from the inlet port passes through a plurality of stages of the thin tube bundle units successively and flows out of the outlet port.

In the present embodiment, for example, a metal material such as stainless steel is preferred as a material constituting the heat transfer thin tube 1. As a material for the housing 4, for example, a resin material such as polycarbonate resin that is transparent and has excellent fracture strength can be used. As a resin material for forming the seal members 3a-3c, for example, it is desirable to use epoxy resin at a portion contacting the material constituting the heat transfer thin tube 1 (e.g., a metal material), and to use polyurethane resin at a portion interposed between the epoxy resin and the housing 4.

### Embodiment 2

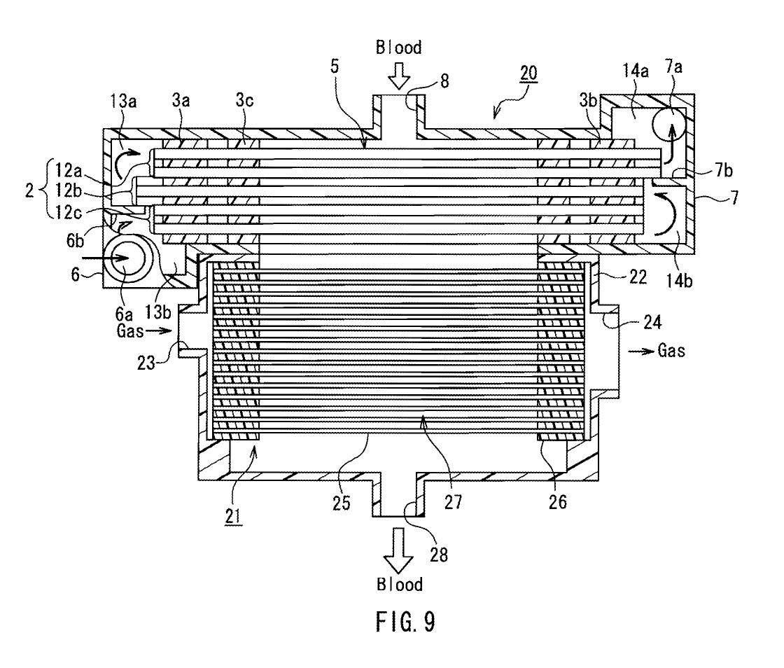

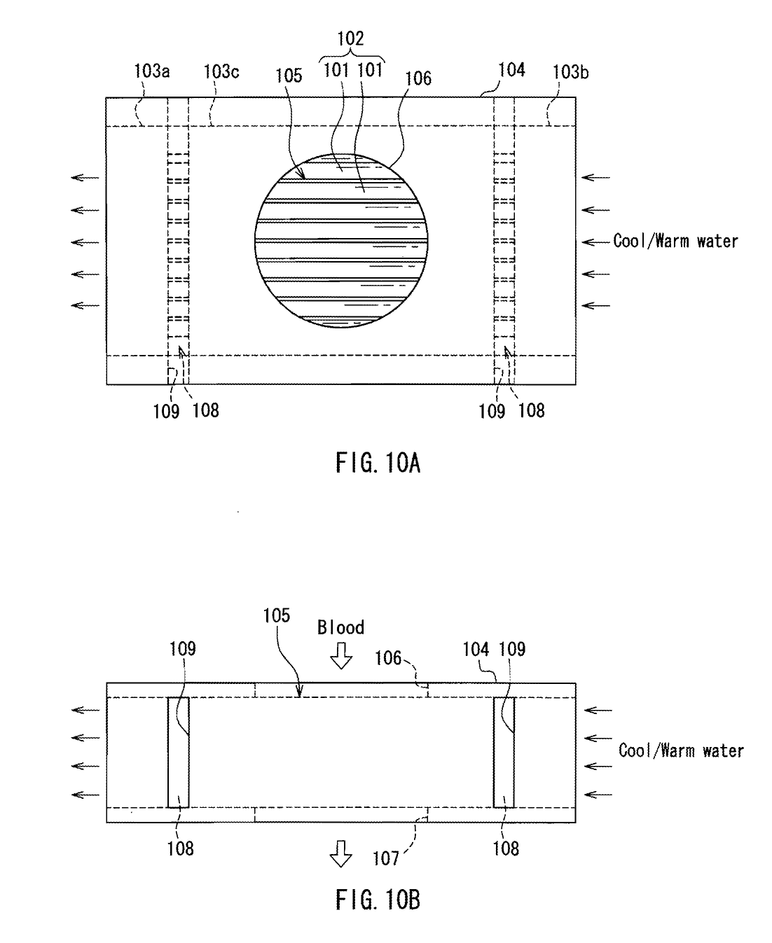

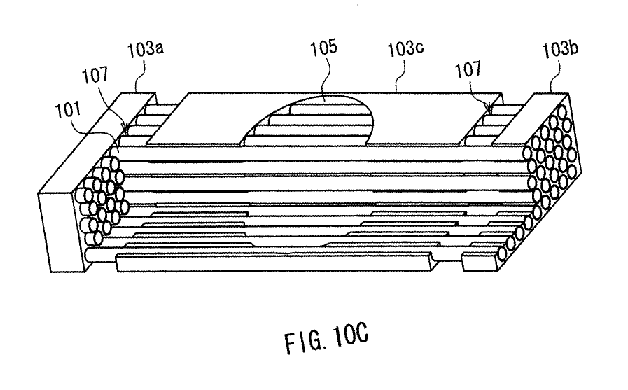

FIG. 9 is a cross-sectional view illustrating an artificial lung device in Embodiment 2. The artificial lung device has a configuration in which a heat exchanger 20 in Embodiment 1 is combined with an artificial lung 21. Note here that the artificial lung device also can have a configuration in which any of the heat exchangers in the above-mentioned other forms is provided instead of the heat exchanger 20.

The heat exchanger 20 is stacked on the artificial lung 21, and the housing 4 of the heat exchanger 20 is connected to a housing 22 of the artificial lung 21. Note here that the housing 4 of the heat exchanger 20 also may be integrated with the housing 22 of the artificial lung 21. In the region of the artificial lung 21, a gas inlet path 23 for introducing oxygen gas and a gas outlet path 24 for discharging carbon dioxide or the like in blood are provided.

The artificial lung 21 includes a plurality of hollow fiber membranes 25 and seal members 26. The seal members 26 seal the hollow fiber membranes 25 so that blood does not enter the gas inlet path 23 and the gas outlet path 24. The seal members 26 seal the hollow fiber membranes 25 in such a manner that both ends of the hollow fibers constituting the hollow fiber membranes 25 are exposed. The gas inlet path 23 and the gas outlet path 24 communicate with each other through the hollow fibers constituting the hollow fiber membranes 25.

Further, the space in which the seal members 26 are not present in the artificial lung 21 constitutes a blood channel 27 in a cylindrical shape, and the hollow fiber membranes 25 are exposed in the blood channel 27. Further, a blood inlet side of the blood channel 27 communicates with an outlet side of the blood channel 5 of the heat exchanger 20.

With the above-mentioned configuration, the blood introduced from the blood inlet port 8 and subjected to heat exchange through the blood channel 5 flows in the blood channel 27 and comes into contact with the hollow fiber membranes 25. At this time, oxygen gas flowing through the hollow fiber membranes 25 is taken in the blood. Further, the blood with oxygen gas taken therein is discharged outside through the blood outlet port 28 provided at the housing 22 and returned to a patient. On the other hand, carbon dioxide in the blood is taken in the hollow fiber membranes 25, and thereafter, is discharged through the gas outlet path 24.

Thus, in the artificial lung device illustrated in FIG. 9, the temperature of the blood is adjusted by the heat exchanger 20, and the blood with the temperature adjusted is subjected to gas exchange by the artificial lung 21. Further, at this time, even if seal leakage occurs in the heat exchanger 20, and the cool/warm water flowing through the heat transfer thin tubes 1 flows out, the cool/warm water appears in the gaps 10, and hence, the leakage can be detected. Therefore, the artificial lung device illustrated in FIG. 9 can detect seal leakage, and the contamination of blood by the cool/warm water can be suppressed.

### INDUSTRIAL APPLICABILITY

According to the present invention, since the flow velocity of the cool/warm water flowing through heat transfer thin tubes can be increased, the heat exchange efficiency can be enhanced while the film resistance in the inner wall of the heat transfer thin tubes is reduced to suppress the increase in volume in the heat exchange region. Thus, the present invention is useful as a medical heat exchanger used in an artificial lung device or the like.

### DESCRIPTION OF REFERENCE NUMERALS

1, 101 heat transfer thin tube

2, 102 thin tube bundle

3a-3c, 103a-103c seal member

4, 104 housing

5, 105 blood channel

6 cool/warm water inlet header

6a cool/warm water inlet port

6b, 7b partition wall

7 cool/warm water outlet header

7a cool/warm water outlet port

8, 106 blood inlet port

9, 107 blood outlet port

10, 108 gap

11, 109 leaked liquid discharge hole

12a-12c first to third thin tube bundle units

13a, 14a upper flow compartment

13b, 14b lower flow compartment

15a-15d protruding portion

16a-16d thin tube row holding member

17 thin tube receiving concave portion

18 tapered face

20 heat exchanger

21 artificial lung

22 housing

23 gas inlet path

24 gas outlet path

25 hollow fiber membrane

26 seal member

27 blood channel

28 blood outlet port

1. A medical heat exchanger, comprising:
a thin tube bundle in which a plurality of heat transfer thin tubes for allowing heat medium liquid to flow through a lumen are arranged and stacked;
a seal member sealing the thin tube bundle while allowing both ends of the heat transfer thin tubes to be exposed and forming a blood channel that crosses the heat transfer thin tubes for allowing blood to flow therethrough so that the blood comes into contact with an outer surface of each of the heat transfer thin tubes;
a housing containing the seal member and the thin tube bundle and provided with an inlet port and an outlet port for the blood positioned respectively at both ends of the blood channel; and
a pair of heat transfer thin tube headers forming flow chambers that respectively contain both ends of the thin tube bundle and having an inlet port and an outlet port for the heat medium liquid,
wherein the thin tube bundle is divided into a plurality of stages in a flow direction of the blood channel, and functions as a stack structure of thin tube bundle units of the respective stages, each stage being composed of members of the plurality of the heat transfer thin tubes,
at least one of the flow chambers is partitioned, by a partition wall provided so as to correspond to a border between the thin tube bundle units, into a plurality of flow compartments so that each flow compartment contains an end of one or two stages of the thin tube bundle units, whereby a channel is formed such that the heat medium liquid flowing in from the inlet port is introduced via any one of the flow compartments so as to pass through the plurality of stages of the thin tube bundle units successively and flows out of the outlet port via another of the flow compartments, and
an end of one of the thin tube bundle units that is positioned on both sides of the border corresponding to the partition wall protrudes further than an end of the other thin tube bundle unit, and a side face of the partition wall contacts an side face of the protruding thin tube bundle unit, whereby the flow compartments on both sides of the partition wall are separated from each other.

2. The medical heat exchanger according to claim 1, wherein, of the thin tube bundle units of the stages on the both sides of the border corresponding to the partition wall, an end of the thin tube bundle unit placed on a side where the heat medium liquid is introduced in the channel of the heat medium liquid protrudes further than an end of the thin tube bundle unit placed on a side where the heat medium liquid is discharged.

3. The medical heat exchanger according to claim 1, wherein a side face portion of the partition wall contacting a side face of the thin tube bundle unit forms a taper, which is made thinner toward an inside of the heat transfer thin tubes.

4. The medical heat exchanger according to claim 1, wherein the heat transfer thin tube headers are formed so that the heat medium liquid successively passes from the thin tube bundle unit in a lower stage placed on a downstream side of the blood channel to the thin tube bundle unit in an upstream stage placed on an upstream side.

5. The medical heat exchanger according to claim 1, wherein the blood channel is formed in a cylindrical shape whose circumference is sealed with the seal member.

6. An artificial lung device, comprising:
the heat exchanger according to claim 1; and
an artificial lung having a blood channel that crosses a gas channel so as to perform gas exchange,
wherein the heat exchanger and the artificial lung are stacked, and the blood channel of the heat exchanger and the blood channel of the artificial lung communicate with each other.
